(19) United States
(12) Patent Application Publication
(10) Pub. No.: US 2024/0035002 A1
(43) Pub. Date: Feb. 1, 2024

(54) METHOD FOR PURIFYING VIRUS

(71) Applicant: Oxford University Innovation Limited, Oxford (GB)

(72) Inventors: Alexander DOUGLAS, Oxford (Oxfordshire) (GB); Carina Citra Dewi JOE, Oxford (Oxfordshire) (GB)

(21) Appl. No.: 18/039,818

(22) PCT Filed: Dec. 10, 2021
(86) PCT No.: PCT/EP2021/085238

§ 371 Date: Jun. 1, 2023

(30) Foreign Application Priority Data
Dec. 10, 2020 (GB) 2019454.4

(51) Int. Cl. C12N 7/02; A61K 39/235; B01D 15/36; B01D 61/58; C12N 9/16

(52) U.S. Cl. CPC C12N 7/02; A61K 39/235; B01D 15/363; B01D 61/58; C12N 9/16; C12Y 301/30002; A61K 2039/5256

(57) ABSTRACT

The invention relates to a method for purifying an adenovirus comprising (a) providing a liquid sample comprising adenovirus, (b) clarifying said sample by depth filtration, (c) performing anion exchange chromatography comprising the steps of (i) directly applying the clarified sample of (b) to an anion exchange column, (ii) eluting adenovirus from the anion exchange column to provide an eluate. The invention also relates to adenovirus produced from said methods, and to compositions comprising same.

### FIELD OF THE INVENTION

The invention relates to a method for purifying virus such as adenovirus. In particular the invention relates to a method for purifying virus such as adenovirus from a cultured production system.

### BACKGROUND

Adenovirus vectored vaccines are among the leading approaches to vaccine development such as SARS-CoV-2 vaccine development. A perceived disadvantage, particularly in comparison to DNA and RNA vaccine platforms, is the complexity of adenovirus manufacturing. This typically involves a fed-batch or perfusion upstream process (USP), followed by a multi-step downstream process (DSP; most commonly depth filter clarification, tangential flow filtration [TFF], anion exchange chromatography [AEX], and a second TFF step) (Vellinga, J., et al., Challenges in manufacturing adenoviral vectors for global vaccine product deployment. Hum Gene Ther, 2014. 25(4): p. 318-27). The invention addresses problem(s) associated with large-scale process development and technology transfer to multiple manufacturing sites, seeking to improve upon the yield and scalability of previously reported processes (e.g. Fedosyuk et al 2019 Vaccine vol 37 pages 6951-6961—see below).

Typical adenovirus downstream processes involve depth-filter clarification, a tangential flow filtration (TFF) concentration/diafiltration step (with a relatively high molecular weight cut-off membrane, such that a large proportion of the lower MW protein & nucleic acid impurities are cleared in the permeate), an anion exchange (AEX) step, and a final formulation step (size exclusion chromatography or TFF). Such known processes have been reviewed (Vellinga J, Smith J P, Lipiec A, Majhen D, Lemckert A, van Ooij M, et al. Challenges in manufacturing adenoviral vectors for global vaccine product deployment. Hum Gene Ther. 2014; 25(4):318-27). The inclusion of the first TFF concentration/diafiltration step has proven necessary to achieve adequate product recovery and host cell protein reduction in these prior art methods.

Fedosyuk et al 2019 (Vaccine vol 37 pages 6951-6961 (doi: 10.1016)) discloses simian adenovirus vector production for early-phase clinical trials. A simple method applicable to multiple serotypes and using entirely disposable product-contact components is taught. This document addresses the problem of small or early-stage production. For example it is stated in the abstract “There is a need for rapid production platforms for small GMP batches of non-replicating adenovirus vectors for early-phase vaccine trials, particularly in preparation for response to emerging pathogen outbreaks.” The authors describe the use of small quantities of experimental purification media (NatriFlo® HD-Q). The authors conclude that 2-4 Litre process size is comfortably adequate for their purposes. Moreover, the purification process is consistently always described with an essential first Tangential Flow Filtration (TFF) step preceding ion exchange chromatography (IEX) (anion exchange chromatography (AEX)). See for example section “3.4 Selection of downstream process media” on page 6956 of Fedosyuk et al. The authors even validate the performance of this first TFF step by reference to known methods previously reported using 300 kDa cutoff TFF devices to pre-filter the sample before IEX. Thus it is clear that this initial TFF step before IEX is essential in Fedosyuk et al, and indeed known methods more generally.

Crucell's known perfusion-based process is disclosed in WO2011/098592, which describes a method for producing recombinant adenovirus serotype 26 (rAd26), the method comprising culturing producer cells in suspension with a perfusion system, and infecting said cells at a density of between 10e6 viable cells/mL and 16e6 viable cells/mL. This method involves an alternating tangential flow (ATF) perfusion system. This method is complicated and can be expensive. Also this process is challenging to transfer to manufacturing partners. Also this process achieves disadvantageous ratios of product to cells.

U.S. Pat. No. 8,124,106B2 discloses a method for removing certain adenovirus proteins from a preparation of subgroup B recombinant human adenovirus particles, in particular removing the proteins comprising subgroup B adenoviral proteins II, III, IV, and 52.55 k.

U.S. Pat. No. 8,574,595B2 discloses a method for purification of a recombinant adenovirus, which method is based on at least two ultrafiltration steps followed by a sterile filtration step. Other known non-perfusion processes in the art obtain limited yields such as ˜200 doses per litre (Vaccitech/Advent) or ˜1000 doses per litre (Tianjin Cansino).

The present invention seeks to overcome problem(s) associated with the prior art.

### SUMMARY

To the inventors' knowledge, there has not previously been a report of successful purification of adenovirus, to the standards required for clinical use, in a process with only depth filter clarification, AEX and a formulation step.

In more detail, prior art techniques such as Fedosyuk et al 2019 require use of a Pre-AEX TFF (sometimes referred to as ‘TFF1’). In other words known methods require a tangential flow filtration step before ion exchange chromatography such as anion exchange chromatography. These known techniques have drawbacks such as needing large volumes to be handled. This is cumbersome and also requires large volumes of costly buffer.

In contrast the inventors teach the removal of this Pre-AEX TFF step. In particular the inventors teach the direct application of clarified liquid sample to the AEX column (AEX membrane). This is a departure from the known techniques which have relied on complex filtration before AEX.

As is explained in more detail below, the inventors also teach load and wash salt concentrations useful to maximise retention/recovery and purification using this AEX technique. The inventors teach how to exploit charge characteristics of the viral capsid in order to achieve these time- and cost-saving advantages.

Thus in one aspect the invention provides a method, such as a method for purifying an adenovirus, said method comprising
(a) providing a liquid sample comprising adenovirus
(b) clarifying said sample by filtration, such as depth filtration,
(c) performing anion exchange chromatography comprising the steps of
   (i) directly applying the clarified sample of (b) to an anion exchange column
   (ii) eluting adenovirus from the anion exchange column to provide an eluate.

Suitably ‘directly’ has its normal meaning in the art. For example ‘directly’ means without intervening steps. So for example “directly applying the clarified sample of (b) to an anion exchange column” means that the clarified sample of (b) undergoes no further transformative processing step before being applied to the anion exchange column. Of course the clarified sample may need to be transported/pumped. Of course the clarified sample may need to be adjusted e.g. the salt concentration may need to be adjusted e.g. the pH may need to be adjusted but the clarified sample would not undergo a substantive processing step such as TFF before being applied to the anion exchange column. Suitably a TFF step before application to the anion exchange column is omitted. Suitably a TFF step before application to the anion exchange column is specifically excluded.

Suitably the anion exchange chromatography of (c) comprises membrane anion exchange chromatography.

Suitably the membrane comprises quaternary amines.

More suitably the membrane comprises Sartobind® Q membrane.

The anion exchange may include prefilter wherein the clarified liquid is passed through the prefilter before being applied to the anion exchange membrane or column. Such a prefilter is included as part of the anion exchange process such that the clarified sample is applied directly to anion exchange. Use of a prefilter can be used to remove particulates to avoid fouling of the AEX membrane or column. Such a prefilter may have a pore size of 0.15 μm-0.5 μm filter, such as a pore size of about 0.2 μm.

Suitably the anion exchange chromatography is carried out with load and wash salt concentrations in the range 24 to 31 mS/cm.

More suitably the anion exchange chromatography is carried out with load and wash salt concentrations in the range 24 to 31 mS/cm, with 20 mM Tris pH 8.0.

More suitably the anion exchange chromatography is carried out with load salt concentrations in the range 24 to 25 mS/cm, wash salt concentrations in the range 27 to 28 mS/cm, and an elution salt concentrations in the range 39 to 40 mS/cm.

Suitably the anion exchange chromatography is carried out with load and wash salt concentrations in the range 280 mM NaCl to 361 mM NaCl.

More suitably the anion exchange chromatography is carried out with load and wash salt concentrations in the range 280 mM NaCl to 361 mM NaCl, with 20 mM Tris pH 8.0.

Suitably the anion exchange chromatography is carried out with wash conditions of 15 mS/cm to 30 mS/cm conductivity.

More suitably the anion exchange chromatography is carried out with wash conditions of 15 mS/cm to 30 mS/cm conductivity, with 20 mM Tris pH 8.0.

Suitably the sample is adjusted to a conductivity of 28 mS/cm before applying to the anion exchange column.

Suitably the sample is adjusted to a conductivity of 28 mS/cm, and to pH 8.0, before applying to the anion exchange column.

Suitably said depth filtration of step (b) comprises primary clarification followed by secondary clarification.

Suitably said depth filtration of step (b) comprises combined primary and secondary clarification.

Suitably said depth filtration of step (b) comprises use of a CoSP depth filter.

Suitably the anion exchange chromatography of (c) is performed in-line with the clarifying depth filtration of (b).

More suitably the anion exchange chromatography of (c) is performed in-line with the clarifying depth filtration of (b) as a single unit operation.

Suitably said liquid sample comprises cell lysate produced from cultured host cells comprising adenovirus.

Suitably said liquid sample consists essentially of cell lysate produced from cultured host cells comprising adenovirus.

Suitably said liquid sample consists of cell lysate produced from cultured host cells comprising adenovirus.

Suitably said cell lysate is produced by treating a sample of cultured host cells comprising adenovirus with a detergent. The sample of cultured host cells comprising adenovirus may comprise cell culture media, wherein the detergent lysis is performed in the cell culture media.

Suitably lysate is prepared as in (Fedosyuk et al 2019—“Simian adenovirus vector production for early-phase clinical trials: A simple method applicable to multiple serotypes and using entirely disposable product-contact components” Vaccine vol 37 pages 6951-6961 (doi: 10.1016)).

Lysis

Lysis is suitably carried out in any suitable manner known to the skilled worker.

For example for lysis a detergent such as Triton X100 may be used, more suitably a detergent which is GMP-suitable (GMP-good manufacturing practice). Suitably, the detergent lysis is performed in cell culture media.

For example lysis may be carried out by subjecting the cells to freeze-thaw cycle(s).

For example lysis may be carried out using a mechanical process.

Lysis is suitably carried out 42 h after infection.

Lysis is suitably initiated by addition of 1/9 culture volume of buffer containing 10% v/v polysorbate 20, 50% w/v sucrose, 20 mM MgCl2, 500 mM Tris pH 8.0 (all from Merck). Suitably nuclease may also be added at this stage, such as Benzonase® (from Merck) to a final in-culture concentration of 60 units/mL, more suitably 15 units/mL. DO (Dissolved Oxygen) and pH control of the bioreactors are suitably de-activated, but agitation and heating to 37° C. is suitably continued.

Incubation to allow lysis is suitably for 2 hours.

Suitably said cell lysate is produced by treating a sample of cultured host cells comprising adenovirus, or a sample of lysed cultured host cells comprising adenovirus, or a sample of cultured host cells comprising adenovirus undergoing lysis, with a nuclease.

Suitably the nuclease is non-specific.

Suitably the nuclease is highly-active.

Suitably the nuclease is a DNase/RNase.

Suitably the concentrations and/or timings of addition of nuclease may be varied according to operator needs.

Suitably said nuclease comprises, or consists essentially of, or consists of, DNAse and/or RNAse.

Suitably said nuclease comprises, or consists essentially of, or consists of, endonuclease from Serratia marcescens.

Suitably said nuclease comprises, or consists essentially of, or consists of, Benzonase®.

Suitably said Benzonase® is added to said sample at a final concentration of 10-100 units/millilitre, more suitably 15 units/millilitre.

Suitably, the method does not involve a step of DNA precipitation before depth filtration. The avoidance of a DNA precipitation step simplifies the method making the overall process more efficient and ultimately reducing the resources required, while increasing throughput of the method. The avoidance of a DNA precipitation step also increases the robustness of the method for use in diverse facilities and with diverse adenovirus vector products as it is known that parameters such as precipitation duration and precipitant concentration require optimisation for individual processes and can affect the effectiveness of DNA removal and product recovery in precipitation-based methods.

In another embodiment the invention relates to a method as described above further comprising:
(d) performing buffer exchange on the eluate of (c).

Suitably step (d) comprises exchanging the buffer to A438 formulation buffer (Colloca S, Barnes E, Folgori A, Ammendola V, Capone S, Cirillo A, et al. Vaccine vectors derived from a large collection of simian adenoviruses induce potent cellular immunity across multiple species. Sci Transl Med 2012; 4 (115):115ra2.)

Suitably step (d) comprises tangential flow filtration.

Suitably step (d) is carried out using either PES flat sheet modules (Pellicon® 2 Mini, or Pellicon® XL50 module)(from Merck) or using 300 kDa MWCO PES MidiKros hollow fibre units (from Spectrum Laboratories).

It will be noted that simian Adenoviruses infect non-human primates (e.g. monkeys and/or chimpanzees) rather than humans. Each species contains a mix of human and simian (e.g. chimpanzee) viruses.

Suitably said adenovirus is, or is derived from, a simian adenovirus.

Suitably said adenovirus is, or is derived from, a species E adenovirus.

Suitably said adenovirus is, or is derived from, a species E simian adenovirus.

Suitably said adenovirus is, or is derived from, non-ChAd63 species E adenovirus.

Suitably said adenovirus has an isoelectric point (pI) which is less than two points or less than 1 point different from the pI of ChAdOx1 nCoV-19. The pI of an adenovirus can be routinely determined for example by isoelectric focussing or electrophoretic mobility. Such adenoviruses with a similar pI to ChAdOx1 nCoV-19 would be expected to behave similarly in their binding during AEX, as discussed in the “CHARGE CHARACTERISTICS” section herein.

Even more suitably said adenovirus is ChAdOx1.

Most suitably the adenovirus is ChAdOx1 nCoV-19.

In one embodiment suitably said adenovirus is an adenovirus having a capsid with charge characteristics similar to, or substantially the same as, those of ChAdOx1.

Suitably said adenovirus is an adenovirus having capsid charge characteristics such that its elution conductivity, in 20 mM Tris pH 8 on a Sartobind Q or Pall Mustang Q membrane chromatography unit, exceeds 25 mS/cm.

In another embodiment the invention relates to an adenovirus prepared by a method as described above.

In another embodiment the invention relates to a composition comprising an adenovirus prepared by a method as described above.

In one embodiment suitably the composition is a pharmaceutical composition.

In one embodiment suitably composition is a vaccine composition.

In an aspect, the invention provides a method consisting of:
(a) providing a liquid sample comprising adenovirus
(b) clarifying said sample by depth filtration
(c) performing anion exchange chromatography by
   (i) directly applying the clarified sample of (b) to anion exchange
   (ii) eluting adenovirus from the anion exchange to provide an eluate, optionally wherein:
(i) no DNA precipitation is performed prior to the depth filtration, and
(ii) the adenovirus is ChAdOx1, or is an adenovirus that has an isoelectric point (pI) which is less than two points or less than one point different from the pI of ChAdOx1 nCoV-19.

In an aspect, the invention provides a method consisting of:
(a) providing a liquid sample comprising adenovirus, wherein said liquid sample comprises cell lysate produced from cultured host cells comprising adenovirus, optionally wherein:
   (i) said cell lysate is produced by treating a sample of cultured host cells comprising adenovirus with a detergent;
   (ii) said cell lysate is treated with a nuclease, such as benzonase; and/or
   (iii) the adenovirus is ChAdOx1, or is an adenovirus that has an isoelectric point (pI) which is less than two points or less than one point different from the pI of ChAdOx1 nCoV-19;
   (iv) no DNA precipitation has been performed on the sample;
(b) clarifying said sample by depth filtration
(c) performing anion exchange chromatography by
   (i) directly applying the clarified sample of (b) to anion exchange
   (ii) eluting adenovirus from the anion exchange to provide an eluate.

In an aspect, the invention provides a method of manufacturing a vaccine formulation comprising or consisting of:
(a) providing a liquid sample comprising an adenovirus, wherein said liquid sample comprises cell lysate produced from cultured host cells comprising the adenovirus, optionally wherein:
   (i) said cell lysate is produced by treating a sample of cultured host cells comprising the adenovirus with a detergent;
   (ii) said cell lysate is treated with a nuclease, such as benzonase;
   (iii) no DNA precipitation has been performed on the sample; and/or
   (iv) the adenovirus is ChAdOx1, or is an adenovirus that has an isoelectric point (pI) which is less than two points or less than one point different from the pI of ChAdOx1 nCoV-19
(b) clarifying said sample by depth filtration
(c) performing anion exchange chromatography by
   (i) directly applying the clarified sample of (b) to anion exchange membrane after the clarified sample has been passed through a prefilter, optionally when the prefilter has a pore size of about 0.2 μm;
   (ii) eluting adenovirus from the anion exchange to provide an eluate;
(d) performing buffer exchange on the eluate of (c) by comprises tangential flow filtration to provide a vaccine formulation.

### DETAILED DESCRIPTION

The invention relates to an adenovirus vector manufacturing process. The process has been demonstrated at 3 litre (3 L) scale.

A cGMP-ready 200 L-scale (˜1 m dose) manufacturing process has subsequently been developed. This finds application in producing (e.g.) ChAdOx1 nCoV-19 vaccine at large scale.

ChAdOx1 nCoV-19 means the spike protein of nCoV-19 expressed from the ChAdOx1 vector. In more detail, “ChAdOx1 nCoV-19” means the ChAdOx1 adenoviral vector as described in Dicks et al. (2012) PLoS ONE 7(7): e40385, and/or in WO2012/172277, comprising a nucleotide sequence encoding a 32aa tPA leader fused to SARS-Cov-2 spike protein inserted at the E1 locus of the ChAdOx1 adenoviral vector under the control of the CMV (cytomegalovirus) ‘long’ promoter. In case any further detail is required we refer to van Doremalen et al 2020 “ChAdOx1 nCoV-19 vaccination prevents SARS-CoV-2 pneumonia in rhesus macaques” (bioRxiv preprint document id: https://doi.org/10.1101/2020.05.13.093195).

For the avoidance of doubt, references to ‘upstream’ (US) methods are references to viral production methods before/whilst the virus remains in the culture vessel.

References to ‘downstream’ (DS) methods are references to viral purification methods from the point that the virus is removed from the culture vessel (e.g. harvesting/removing a quantity of culture comprising host cells/virus from the culture vessel).

We describe a highly efficient process for the manufacture of adenovirus vectors. Adenovirus vectors find application in vaccines. The methods described are suitable for cGMP execution. Methods may be carried out at a range of scales up to >10 m doses per batch. It is an advantage that the methods provide very low cost of goods.

The invention finds application in production of adenoviruses for purposes other than vaccine production e.g. production of oncolytic viruses/oncolytic viral vectors.

The purification process described herein may be carried out on a liquid sample comprising virus produced from a combination of cells, preferably HEK293-based, most preferably T-rex cells (which are very good at producing adenovirus, even when the antigen expression is de-repressed), and medium/feed (preferably Fujifilm BalanCD293 medium/feed) which enable upstream process yield of adenovirus >4e14 virus particles (VP) per L of culture (sometimes >1e15 VP/L). This production level may be achieved without requiring perfusion/medium-exchange (i.e. >8000 doses per L from the culture, before purification).

In addition suitably the host cell/antigen repression promoter combination may contribute to the overall efficiency (preferably virus is produced using T-rex & tet-repressing promoter).

The purification process described herein may be carried out on a liquid sample from a production process having a cell-specific productivity of >100,000 VP per cell (sometimes >200,000 VP per cell).

The purification process described herein may be carried out on a liquid sample comprising cell densities exceeding 2e6 cells/mL.

The purification process described herein may be carried out on a liquid sample having the cell-specific productivity noted above (i.e. high ratio of product to contaminants).

The purification process described herein provides a simplified, rapid & highly cost-effective harvest and purification process. Suitably the invention relates to a method comprising only a nuclease step, a depth filter for clarification step, a single chromatography step (anion exchange, in-line with the clarification filter) and a single tangential flow filtration step. Most suitably the steps are carried out in that order.

The inventor asserts a yield >2e15 VP per L of culture, using perfusion, with cell density <1e7 cells/mL.

Suitably the adenovirus comprises a AdHu5 E4Orf6 substitution. Suitably the adenovirus comprises ChAdOx1, ChAdOx2, or ChAd63.

Suitably the method comprises only a single tangential flow filtration step.

Suitably the method comprises only two unit operations.

Suitably the method comprises only a single chromatography step. Suitably said single chromatography step comprises anion exchange. Suitably this single chromatography step is carried out in-line with the clarification filter.

Suitably when the adenovirus comprises an antigen of interest, the antigen-repressing cell/promoter combination comprises HEK293/Tet+CMV long.

The invention provides the advantage of reducing the amount of nuclease, such as expensive Benzonase® nuclease, which is needed. This advantage is facilitated by the relatively low cell density as compared to (e.g.) known perfusion-based processes such as Johnson & Johnson's perfusion-based process.

The invention finds application in manufacture of adenovirus-vectored vaccines.

The invention substantially increases the amount of adenovirus produced per litre of culture.

The invention makes it easier to purify the virus (for example because there is a high ratio of product to cells at the start).

The process makes it possible to obtain ˜4000 doses of vaccine per L of culture. A known processes such as J&J's perfusion-based process may achieve a similar level but is more complicated & expensive & harder to transfer to manufacturing partners, and does not achieve such favourable ratios of product to cells.

Thus the inventor asserts that the process of the invention obtains 4×-20× the yields of known methods such as:
˜200 doses per litre (Vaccitech/Advent)
˜1000 doses per litre (Tianjin Cansino)

The process of the invention makes it possible to obtain ˜8000 doses of vaccine per L of culture in a cost-effective and simplified manner.

Thus the inventor asserts that the process of the invention obtains 8×-40× the yields of known methods such as:
˜200 doses per litre (Vaccitech/Advent)
˜1000 doses per litre (Tianjin Cansino)

This provides benefits such as making the economics of virus/viral vector production significantly more favourable. This also makes it possible to prepare larger quantities of virus/viral vector more rapidly, which is important in an emergency situation (e.g. a pandemic of a life-threatening virus) and/or if first to market with such a product.

The inventor asserts that the method of the invention is the first Adenovirus production process to achieve these levels of productivity without drawback(s) of the prior art.

Suitably the virus being produced (purified) as described herein comprises the AdHu5 E4orf6. ChAdOx1, ChAdOx, and ChAd63 each carry this AdHu5 E4orf6.

As noted above, to the inventors' knowledge, there has not previously been a report of successful purification of adenovirus, to the standards required for clinical use, in a process with only depth filter clarification, AEX and a formulation step.

For the avoidance of doubt, ‘standards required for clinical use’ may refer to the levels of HCP (host cell protein) in the purified adenovirus product; suitably the method of the invention delivers mean levels of <1000 ng of HCP per human dose, more suitably the method of the invention delivers mean levels of <200 ng of HCP per human dose. Suitably the adenovirus product of the invention comprises mean levels of <1000 ng of HCP per human dose, more suitably the adenovirus product of the invention comprises mean levels of <200 ng of HCP per human dose.

E4orf6

In principle any E4orf6 from a simian adenovirus may be used. However it must be noted that for correct function the E4orf6 has to interact with the Ad5 E1 protein (expressed by the host cell) so the E4orf6 needs to be sufficiently similar to Ad5 E4orf6 to support this functional interaction with Ad5 E1.

Suitably the E4orf6 is Ad5 E4orf6. Ad5 means Human adenovirus C serotype 5 (HAdV-5) (Human adenovirus 5).

Suitably E4orf6 comprises the amino acid sequence of accession number Uniprot Q6VGT3 (E4orf6 protein sequence).

Suitably nucleotide sequence encoding E4orf6 corresponds to the E4orf6 coding sequence of accession number Genbank NC_001405.1 (whole adeno genome sequence encoding E4orf6) (E4orf6 coding sequence 33193 to 34077).

This has the advantage is that species E Chimp Ad E4orf6's fail to interact with the Ad5 protein.

AEX Load/Wash

Buffer conditions are suitably with 20 mM Tris pH 8.0 unless otherwise stated.

Suitably load and wash salt concentrations are in the range 24 to 31 mS/cm conductivity, with 20 mM Tris pH 8.0.

Load and wash conditions may be expressed as salt concentrations rather than conductivity. With this buffer (20 mM Tris pH 8.0), 24 mS/cm is circa. 280 mM NaCl.

Suitably load and wash salt concentrations are in the range 280 mM NaCl to 361 mM NaCl (20 mM Tris pH 8.0).

In case any further guidance is required, the table shows Conductivity to Salt Concentration Conversion (20 mM Tris pH 8.0):

| Conductivity | Salt concentration |
|---|---|
| 24 mS/cm | 280 mM NaCl |
| 28 mS/cm | 320 mM NaCl |
| 30 mS/cm | 350 mM NaCl |
| 31 mS/cm | 361 mM NaCl |

In one embodiment the anion exchange chromatography is carried out with wash conditions of up to 30 mS/cm conductivity, with 20 mM Tris pH 8.0. Here ‘up to’ infers a lower value below 30 mS/cm conductivity at the bottom of the range. The lower value is suitably the conductivity of the culture medium without addition of salt. In one embodiment the anion exchange chromatography is carried out with wash conditions of (the conductivity of the culture medium without addition of salt) up to (30 mS/cm) conductivity.

The conductivity of the culture medium without addition of salt may vary according to the medium used or other variables specific to an individual run (e.g. how much sodium bicarb had been added to adjust pH).

The conductivity of the culture medium without addition of salt would typically be 15-20 mS/cm.

Suitably load and wash salt concentrations are in the range 15-20 mS/cm to 30 mS/cm (20 mM Tris pH 8.0).

Suitably load and wash salt concentrations are in the range 15 mS/cm to 30 mS/cm (20 mM Tris pH 8.0).

Suitably load and wash salt concentrations are in the range 20 mS/cm to 30 mS/cm (20 mM Tris pH 8.0).

Liquid Sample

The liquid sample is suitably a quantity of liquid comprising host cells/virus. Suitably said liquid comprises medium from the culture vessel in which the host cells/virus were being incubated or cultured. Suitably the liquid sample comprises medium directly removed from the culture vessel (e.g. directly harvested from the bioreactor/fermenter/culture chamber). Thus, the liquid sample comprises the medium containing the cells and/or material derived from cell death or lysis).

In one embodiment suitably the liquid sample is treated before clarification. Treatment of the liquid sample before clarification may comprise addition of detergent to lyse the cells. The use of detergent to lyse the cells is advantageous because it is highly scalable, unlike other methods of lysis, such as freeze-thaw mediated lysis.

Treatment of the liquid sample before clarification may comprise addition of nuclease to digest nucleic acid.

Optionally a centrifugation and/or gravity-settling step may be carried out prior to clarification.

Suitably the liquid sample comprises 10 to 2000 Litres.

The liquid sample volume is sometimes referred to as process size.

Suitably the liquid sample comprises, or is derived from, a combination of cells, preferably HEK293-based, and medium/feed such as Fujifilm BalanCD293 medium/feed.

Suitably the liquid sample comprises >2e14 virus particles (VP) per L of culture.

Suitably the liquid sample comprises >1e15 virus particles (VP) per L of culture.

Suitably the liquid sample does not comprise, or is not derived from, perfusion/medium-exchange culture.

Suitably the liquid sample comprises a species E virus (species E adenovirus).

Suitably the liquid sample comprises, or is derived from, a cell-specific productivity of >100,000 VP per cell (sometimes >200,000 VP per cell).

Suitably the liquid sample comprises, or is derived from, cell densities exceeding 2e6 cells/mL.

Suitably the method has a yield >2e15 VP per L of culture, using perfusion, with cell density <1e7 cells/mL.

Suitably the liquid sample comprises any suitable number of cells per mL. It may be that for high cell densities (e.g. where high means >=4e6 cells/mL) that certain optimisations may be helpful. High loading values may present different technical issues. In case of any problem caused by high loading values, the skilled operator may simply dilute the liquid sample. In one embodiment suitably the liquid sample comprises <=4e6 cells/mL. In one embodiment suitably the liquid sample comprises <4e6 cells/mL.

In one particular embodiment the skilled operator may load a smaller amount of the sample per unit of AEX membrane.

In one particular embodiment the skilled operator may pre-treat the lysate with a centrifugation, filtration or chromatography step suitable to remove substances which might otherwise compete with the product for binding to the AEX membrane.

In some embodiments, the liquid sample comprising adenovirus is a whole cell lysate in cell culture medium, wherein lysis is performed using detergent-mediated lysis. This liquid sample may then be directly subjected to clarification by depth filtration without any intervening purification or clarification steps, such as DNA precipitation. The liquid sample may, however, be treated with a nuclease, such as benzonase, as disclosed elsewhere herein.

Clarification of Liquid Sample

Clarification is suitably initiated two hours after addition of lysis buffer (i.e. initiation of lysis/addition of detergent).

Clarification has its normal meaning in the art and refers to removal of impurities such as cells and/or cell debris from the liquid sample.

In one embodiment clarification of step (b) is by use of any suitable method which may include centrifugation or filtration.

In one embodiment clarification is by use of centrifugation.

Thus in one embodiment the invention relates to a method for purifying an adenovirus comprising
(a) providing a liquid sample comprising adenovirus
(b) clarifying said sample by depth filtration or another suitable method which may include, or consist of, centrifugation
(c) performing anion exchange chromatography comprising the steps of
(i) directly applying the clarified sample of (b) to an anion exchange column
(ii) eluting adenovirus from the anion exchange column to provide an eluate.

Suitably clarification does not involve tangential flow filtration. Suitably tangential flow filtration is not used for clarification in the method of the invention. Suitably tangential flow filtration is omitted before ion exchange chromatography. Suitably tangential flow filtration is specifically excluded before ion exchange chromatography.

Suitably clarification is by use of filtration.

Suitably clarification is by use of depth filtration.

Suitably clarification is by use of a depth filter.

Depth filtration is used to remove a broad range of particles or impurities, such as whole (unlysed) cells, cell debris or other material.

A depth filter may not have a defined pore size or structure. Particles are entrapped or adsorbed both within and on the filter due to a ‘random’ matrix or structure which creates a complex path through the filter. When a depth filter does have a nominal size cutoff or pore size, suitably a nominal micron rating of 0.2 to 2 μm is used. Thus suitably the nominal micron rating of the CoSP filter is 0.2 to 2 μm.

Depth filter cartridges/filtration materials are made of carefully selected materials such as polypropylene, cotton or glass fibre to physically detain and thereby remove these particles. More typically for bioproduction applications, depth filtration materials may comprise a fibrous bed of cellulose or polypropylene fibres.

In one embodiment suitably clarification is by use of a CoSP depth filter.

Most suitably clarification is by use of a Millistak+® Merck/Millipore “Millistak® HC Pro Synthetic Depth Filter” CoSP Series, 23 cm² Catalogue Number MCoSP23CL3 as available from Merck KGaA, Darmstadt, Germany; available Worldwide via SigmaAldrich/Merck Life Science UK Limited, The Old Brickyard, New Rd, Gillingham, Dorset, SP8 4XT, U.K.

AEX Membrane

Suitably the AEX is carried out using a strong anion exchange column.

Suitably the AEX is carried out using an AEX membrane.

Suitably the AEX is carried out using an AEX membrane comprised by a strong anion exchange column.

Suitably the AEX membrane comprises functional groups. Suitably said functional groups are quaternary amines. Suitably the AEX membrane comprises quaternary amines.

Suitably the AEX membrane comprises a material wherein the functional groups are quaternary amines.

Suitably the AEX membrane comprises Sartobind® Q membrane.

Suitably the Sartobind® Q membrane is from Sartorius AG, Germany (Sartorius Stedim Biotech GmbH, August-Spindler-Straße 11, 37079 Goettingen, Germany).

There is a large range of products using the same membrane in different sizes. The skilled operator may select a product according to their needs. An example of a most suitable product is 96IEXQ42E9BFF (Sartobind® Q 150 mL, 8 mm bed height) (Sartorius Stedim Biotech GmbH, August-Spindler-Straße 11, 37079 Goettingen, Germany).

Charge Characteristics

The inventors assert that the invention exploits the unexpectedly strong interaction resulting from the negative charge of the capsid protein of the adenovirus. The inventors assert that this unexpectedly strong interaction resulting from the negative charge is not predicted from analysis of the capsid protein sequence. Without wishing to be bound by theory, the inventors believe that this unexpectedly strong interaction resulting from the negative charge of the capsid protein is exploited in the methods of the invention, thereby facilitating the excellent purification achieved by directly applying clarified lysate to the AEX column.

Suitably the adenovirus is an adenovirus having a capsid with charge characteristics similar to those of ChAdOx1.

In some embodiments, the adenovirus is not adenovirus serotype 5. Suitably, the magnitude of the negative charge of the adenovirus' hexon charge is less than that of adenovirus serotype 5, which has a net hexon charge of ˜23.8. In some cases, the magnitude of the negative charge of the adenovirus hexon is smaller than 22, or smaller than 20. In some cases the adenovirus has net negative hexon charge of between 21 and 15.

In one embodiment the invention relates to a method as described above wherein said adenovirus is an adenovirus having a capsid with charge characteristics similar to those of ChAdOx1.

Suitably “charge characteristics similar to those of ChAdOx1” means capsid charge characteristics such that its elution conductivity, in 20 mM Tris pH 8 on a Sartobind Q or Pall Mustang Q membrane chromatography unit (e.g. CLM05MSTGQP1—Mustang® Q chromatography membrane in Novasip™ capsule, 10 mL bed volume, 1½ in sanitary flange connections—from Pall Biotech—United Kingdom, 5 Harbourgate Business Park Southampton Road, Portsmouth PO6 4BQ, Hampshire, United Kingdom), exceeds 25 mS/cm, or preferably 30 mS/cm.

Clarification

A two-step clarification may be used. In this embodiment two filters are used in-line, such as a coarse primary filter and then a finer, smaller-pore secondary filter.

Suitably the depth filtration of step (b) comprises primary clarification followed by secondary clarification.

‘primary clarification’ and ‘secondary clarification’ refer to different pore sizes of filter used for clarification.

Suitably pore size of the primary filter will be greater than the secondary filter.

Suitably pore size of the secondary filter will be greater than 0.1 um.

Suitably neither filter will be capable of binding adenovirus.

Suitably filters based upon diatomaceous earth, cellulose, polyacrylic fibre and/or silica may be used.

In one embodiment the primary filter may be Millistak DoHC or DoSP.

In one embodiment the secondary filter may be CoHC or CoSP.

These filters are commercially available from Merck Life Science UK Limited, Suite 21, Building 6, Croxley Green Business Park, Watford, Hertfordshire WD18 8YH, United Kingdom and/or from SigmaAldrich/Merck Life Science UK Limited, The Old Brickyard, New Rd, Gillingham, Dorset, SP8 4XT, U.K.

Suitably the method of the invention uses combined primary and secondary clarification. Thus, most suitably the method of the invention uses a single step clarification.

More suitably the depth filtration of step (b) comprises use of Merck Millipore CoSP filter.

There is a large range of products using the same filter/membrane in different sizes. The skilled operator may select a product according to their needs. An example of a most suitable product is Merck Millipore CoSP filter catalogue number MCoSP23CL3. Thus, most suitably the depth filtration of step (b) comprises use of Merck Millipore CoSP filter catalogue number MCoSP23CL3 (Merck Life Science UK Limited, Suite 21, Building 6, Croxley Green Business Park, Watford, Hertfordshire WD18 8YH, United Kingdom).

Nuclease

Any suitable nuclease may be used.

Suitably the nuclease is Benzonase®.

Most suitably the nuclease is Merck 1016970010, (Merck Life Science UK Limited, Suite 21, Building 6, Croxley Green Business Park, Watford, Hertfordshire WD18 8YH, United Kingdom).

Buffer Exchange

In one embodiment the invention provides a method as described above further comprising:
(d) performing buffer exchange on the eluate of (c).

This buffer exchange step may be comprise, or consist of, TFF.

“Tangential flow filtration”, “TFF” (also called cross-flow microfiltration), refers to a mode of filtration in which the solute-containing solution passes tangentially across an ultrafiltration membrane and lower molecular weight salts or solutes are passed through the membrane by applying pressure.

Database Release

Sequences deposited in databases can change over time. Suitably the current version of sequence database(s) are relied upon. Alternatively, the release in force at the date of filing is relied upon.

As the skilled person knows, the accession numbers may be version/dated accession numbers. The citeable accession numbers for the current database entry are the same as above, but omitting the decimal point and any subsequent digits.

GenBank is the NIH genetic sequence database, an annotated collection of all publicly available DNA sequences (National Center for Biotechnology Information, U.S. National Library of Medicine 8600 Rockville Pike, Bethesda MD, 20894 USA; Nucleic Acids Research, 2013 January; 41(D1):D36-42) and accession numbers provided relate to this unless otherwise apparent. Suitably the current release is relied upon. More suitably the release available at the effective filing date is relied upon. Most suitably the GenBank database release referred to is NCBI-GenBank Release 239: 15 Aug. 2020.

UniProt (Universal Protein Resource) is a comprehensive catalogue of information on proteins (‘UniProt: a hub for protein information’ Nucleic Acids Res. 43: D204-D212 (2015).). Suitably the current release is relied upon. More suitably the release available at the effective filing date is relied upon. Most suitably, the UniProt consortium European Bioinformatics Institute (EBI), SIB Swiss Institute of Bioinformatics and Protein Information Resource (PIR)'s UniProt Knowledgebase (UniProtKB) Release 2020 May of 7 Oct. 2020 is relied upon.

Viral Particles (VP) vs Infectious Units (IU)

When comparing yields between AdHu5 processes and non-human-adenoviruses, it is important to note that doses are usually defined in terms of virus particles (VP), but yields are sometimes reported in terms of infectious units (IU). The conversion factor between these, the particle: infectivity ratio, is typically 5 to 20 for AdHu5, but 50 to 200 for a simian adenovirus. Even expressed in VP terms, human doses for AdHu5-based vaccines may be higher (e.g. 0.8-1.6e11VP)(e.g. Zhu F C, et al. Safety and immunogenicity of a recombinant adenovirus type-5 vector-based Ebola vaccine in healthy adults in Sierra Leone: a single-centre, randomised, double-blind, placebo-controlled, phase 2 trial. Lancet. 2017; 389(10069):621-8.) than for most simian adenovirus-vectored vaccines (2.5-5e10 VP)(e.g. Morris S J S, et al Simian adenoviruses as vaccine vectors. Future Virology. 2016; 11(9):649-59); in IU terms, AdHu5 doses are thus much higher than non-human adenovirus doses. As a result, particular care needs to be taken in comparing results expressed in IU.

### Further Advantages

Suitably the process size used is larger than 4 L. Suitably the process size is 10 L or more, suitably 50 L or more, suitably 200 L or more, suitably >200 L, suitably 1000 L or more, suitably 1000 L-2000 L, or even more. Suitably process size means volume of culture vessel e.g. volume of bioreactor or more suitably volume of liquid comprising host cells used to produce the adenovirus.

For example regarding the purification method described herein, process size may be taken to be the volume of liquid sample comprising adenovirus of step (a).

Technical differences and/or advantages are delivered by the invention compared to Fedosyuk et al 2019.

For example, Fedosyuk et al disclose use of nuclease at 60 u/mL Benzonase®, whereas suitably the method of the invention uses 15 u/mL Benzonase®. This delivers the advantage of a cost reduction.

Clarification is suitably using a CoSP depth filter.

For example, Fedosyuk et al disclose Membrane AEX with Tangential Flow Filtered lysate. In contrast, the method of the invention requires direct application of clarified lysate to the AEX column, suitably Membrane AEX system. This advantageously eliminates the cost and time of the initial TFF step taught in known methods such as Fedosyuk.

In more detail, the inventors teach Membrane AEX with directly-loaded clarified lysate (operating in defined range of load and wash salt concentrations [24-31 mS/cm with 20 mM Tris pH 8.0, preferably with some addition of the salt to the lysate to reach conductivity 28 mS/cm]. This offers further advantages, such as carrying out the AEX in-line with/as a single unit-operation with clarification. Avoidance/omission of the initial TFF step taught in known methods is also advantageous.

The inventors assert that this simplification/omission and the positive teaching of the direct application of clarified lysate to the AEX system are probably achieved by exploiting charge characteristics of the viral capsid resulting in high affinity for the AEX membrane (such as Sartobind Q membrane). For example the viral particles can remain bound to a Sartobind Q membrane [Sartorius] under conditions of up to 30 mS/cm conductivity with 20 mM Tris pH 8.0.

These methods are generally applicable to other adenovectors (i.e. the methods are suitably not limited to nCoV-19 ChAdOx1). It is possible that ChAdOx2 may not perform as well in the purification methods of the invention, but the inventor assert that there are other vectors which may be identified or engineered with similar charge characteristics to (for example) ChAdOx1, especially nCoV-19 ChAdOx1, to achieve the same or similar performance in purification according to the methods taught herein.

Methods of the invention are useful in purification from high yield lysates from upstream production.

Final TFF (‘buffer exchange’) is suitably carried out after elution from AEX. In principle an alternative means of buffer exchange (i.e. a non-TFF buffer exchange step) could be used.

An advantage of purification methods according to the present invention compared to known methods using TFF before AEX (such as Fedosyuk et al 2019—“Simian adenovirus vector production for early-phase clinical trials: A simple method applicable to multiple serotypes and using entirely disposable product-contact components” Vaccine vol 37 pages 6951-6961 (doi: 10.1016)) is speed and/or practicality of implementation.

Without wishing to be bound by theory, although improvement in product recovery and/or quality might be attained, the inventors assert that the key improvements are speed and/or practicality of implementation.

TFF requires time, equipment, large volumes of buffer, and its inclusion means that the depth filter clarification, TFF and AEX need to be run as a single unit operation. AEX is quick, whether run as a separate operation after the depth filter clarification, or (even quicker) executed as a single operation with the depth filter clarification.

Prior art attempts to carrying out AEX without preceding TFF (i.e. TFF before AEX) got very poor results (e.g. loss of a large proportion of the virus). Without wishing to be bound by theory, the inventors assert that this may be due to the charge characteristics of the viruses used in the particular prior art methods.

The invention provides a simplified purification method.

The invention provides a purification method comprising the step of directly applying clarified lysate to an anion exchange chromatography (AEX) system. The inventor asserts that it is surprising that this was possible and surprising that such excellent purification and/or yields are obtained by performing the method as described.

This direct application of clarified lysate to AEX advantageously permits the purification method to pass cultured host cells/lysate directly from the reactor (e.g. culture vessel) through a filter (e.g. depth filter) and into the AEX system (e.g. AEX column/membrane). It is an advantage of the invention that these steps may be carried out ‘in line’. It is an advantage of the invention that these steps may be carried out as a ‘single unit operation’. It is an advantage of the invention that the harvested cells/lysate may flow straight through the depth filter and into the AEX system.

This provides advantages such as elimination of a holding tank. Suitably the invention does not have a holding step. Suitably a holding step is omitted. Suitably the apparatus does not involve a holding tank.

This provides advantages such as elimination of a TFF step preceding AEX. Suitably the invention does not have a TFF step preceding AEX. Suitably a TFF step preceding AEX is omitted. Suitably the apparatus does not involve a TFF circuit preceding AEX.

This provides the further advantageously of saving time. A TFF step preceding AEX can take several hours. This time is advantageously eliminated from the process of the invention. Known approaches using a TFF step preceding AEX have to carry out this step using the same volume as in the reactor/culture vessel. Thus in known approaches if the culture is 1000 litres, then 1000 litres of TFF buffer are required. The invention delivers the technical advantage of eliminating this costly and time consuming TFF step preceding AEX.

Suitably the method involves only a single AEX step. This has the advantage of further simplifying the process, thus making it more efficient than purification methods involving multiple AEX steps.

Known methods typically rely on ‘orthogonal’ separations i.e. a series of different separation steps. These achieve separation based upon different properties of the product and the impurities.

In known methods typically a TFF step is used before AEX. In known methods this TFF step preceding AEX removes impurities from the clarified lysate. In known methods this TFF step preceding AEX is a physical filtration, for example with a molecular weight (MWt) cutoff of 300 kDa such that all material with a MWt of <300 kDa goes to waste, and the virus is retained. The inventors estimate that known methods use this TFF step preceding AEX to remove approximately 90% of impurities.

The inventors assert that known methods typically cannot deliver sufficiently pure product without this TFF step preceding AEX.

AEX separates based on a different principle i.e. based on charge.

It is a new technical benefit that the method enables ion exchange chromatography (anion exchange chromatography) (AEX) to be carried out without a preceding tangential flow filtration (TFF).

It is a new teaching provided by the invention to rely on charge-based separation alone, i.e. without a TFF step preceding AEX. It is a surprising advance that the method of the invention can exploit charge-based separation to remove sufficient impurities in the purification without a TFF step preceding AEX.

Known techniques can lose 80% of product (retaining 20% of product). It is an advantage of the invention that up to 90% of product is retained (losing only up to 10% of product).

It is an advantage of the invention that modified AEX is used. For example, by changing the salt concentrations relative to known methods (e.g. using increased salt concentrations relative to known methods) the invention reduces the quantity of impurities which are retained on, or associate with, the AEX column/membrane. This is a further technical benefit provided by the invention. We refer to the accompanying figures, such as FIG. 3C, which illustrate this technical benefit.

The inventor asserts that the invention provides the first Ad process to achieve these levels of productivity without perfusion.

The inventor asserts that the invention is the first method to permit a two-unit-operation downstream process (purification process).

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present invention will now be described further, with reference to the accompanying drawings, in which:

FIG. 1 shows graphs, bar charts and a table.

FIG. 2 shows graphs, diagrams and tables.

FIG. 3 shows graphs, tables and a diagram.

### EXAMPLES

50 L and 200 L runs were performed at Pall Biotech, Portsmouth, UK.

All obtained with BalanCD293 medium/feed combination & HEK293 T-rex cells.

### Example 1: Production/Upstream Process

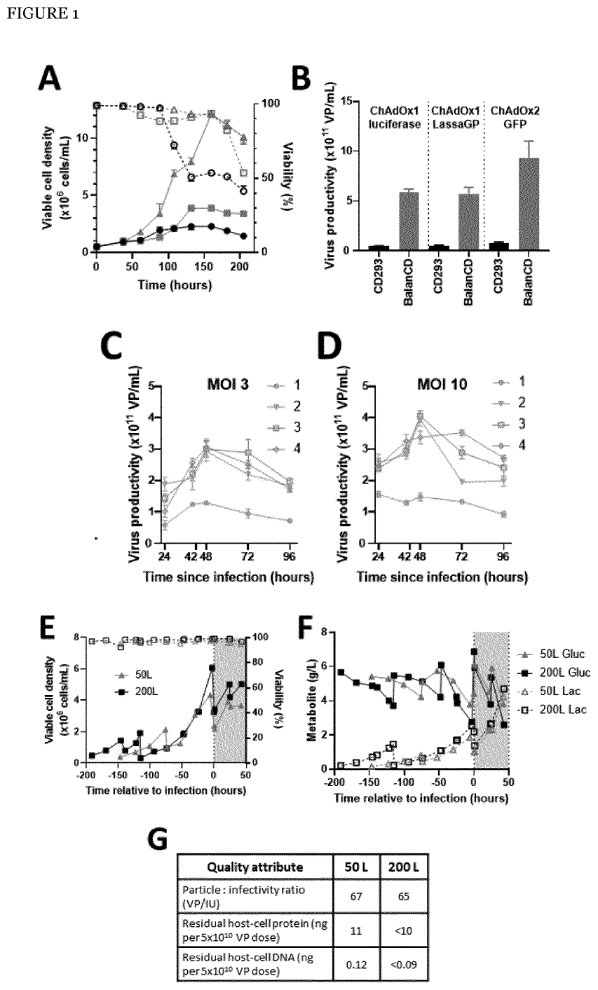

We initially investigated medium/feed combinations for a fed-batch USP. BalanCD293 medium and feed (Fujifilm) was found to support growth of the producer cells (HEK293 T-rex, Thermo) to 1.2×10⁷ cells/mL with high viability (FIG. 1A). Using this combination in small-scale production of adenovirus vectors of two serotypes and carrying three transgenes, we attained productivity exceeding 5×10¹¹ virus particles (VP) per mL, around 5-fold greater than typically obtained in our previous USP (FIG. 1B). To our knowledge such productivity has not previously been reported from a non-perfusion USP: a fall in cell-specific productivity to <1×10⁵VP/cell is commonly observed at cell densities exceeding 1×10⁶ cells/mL (Kamen, A. and O. Henry, Development and optimization of an adenovirus production process. J Gene Med, 2004. 6 Suppl 1: p. S184-92).

Using ChAdOx1 nCoV-19 starting material, we initially investigated the optimal multiplicity of infection (MOI), cell density and time of harvest (FIG. 1C-D). An MOI of 10, cell density of 2-3×10⁶/mL and time of harvest 42-48 hours post infection were selected for further work. This USP was subsequently scaled up, with USP productivities in 10 L, 50 L and 200 L stirred tank reactors (STR) in the range 2-4×10¹¹ VP/mL, and acceptable cell growth and metabolite profiles (FIGS. 1E-F).

Early STR batches made use of a DSP very similar to that we had previously described, which again achieved recovery of 50-60% and quality characteristics compliant with a regulator-accepted specification for product for clinical use (FIG. 1G) (Fedosyuk et al 2019 Vaccine vol 37 pages 6951-6961).

We refer to FIG. 1 which shows development of fed batch process.

Panel A shows cell counts (solid lines, filled symbols) and viability (dashed lines, open symbols) attained during growth of HEK293 T-rex cells in BalanCD medium with (triangles) or without (squares) feed, as compared to the CD293 medium (circles, Thermo) used in our previous process. In the case of the BalanCD medium+feed condition, feed (5% v/v) was added at 36 and 108 hours.

Panel B shows small-scale USP productivity of ChAdOx1-luciferase, ChAdOx1-LassaGP, and ChAdOx2-GFP with BalanCD medium/feed (infected at 4×10⁶ cells/mL, MOI=10) as compared to our previously established conditions in CD293 medium (infected at 1×10⁶ cells/mL, MOI=3) (Fedosyuk et al 2019 Vaccine vol 37 pages 6951-6961).

ChAdOx1-luciferase infections were performed in a 3 L bioreactor; the other two viruses were produced in 30 mL volume in shake flasks.

Panels C-D show small-scale USP productivity of ChAdOx1 nCoV-19 in shake flasks at 30 mL working volume at MOI=3 (C) and MOI=10 (D) respectively. Legend indicates cell density represented by each line, in million cells/mL at point of infection.

Infectious unit (IU) titers broadly paralleled VP titers; results are representative of two replicate experiments.

For panels A-D, points indicate median and error bars show range of results for 2-3 replicate flasks.

Panels E-F: Examples of 50 L and 200 L USP runs (carried out by Pall Biotech). (E) shows cell growth (solid lines) and viability (dashed lines). (F) shows glucose (solid lines) and lactate (dashed lines) concentrations.

Panel G: Examples of quality of drug substance from 50 L and 200 L runs (carried out by Cobra).

### Example 2: Purification/Downstream Process

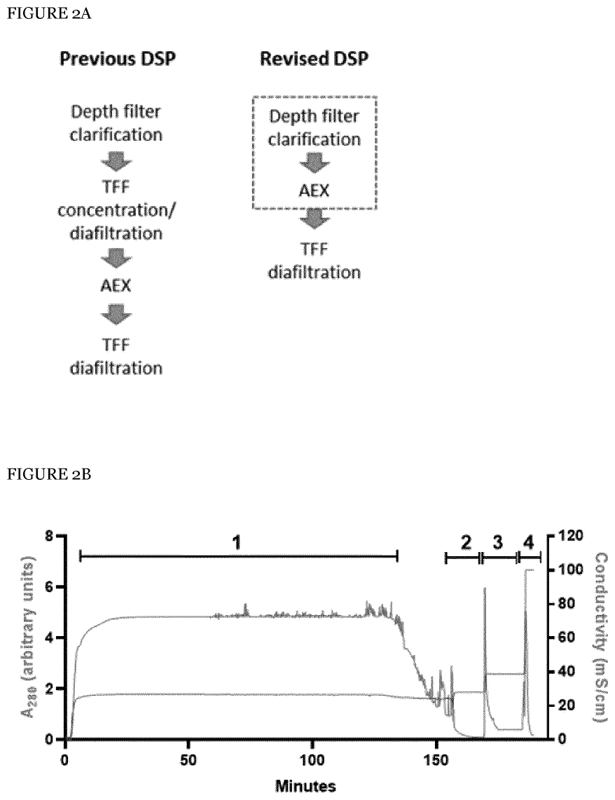

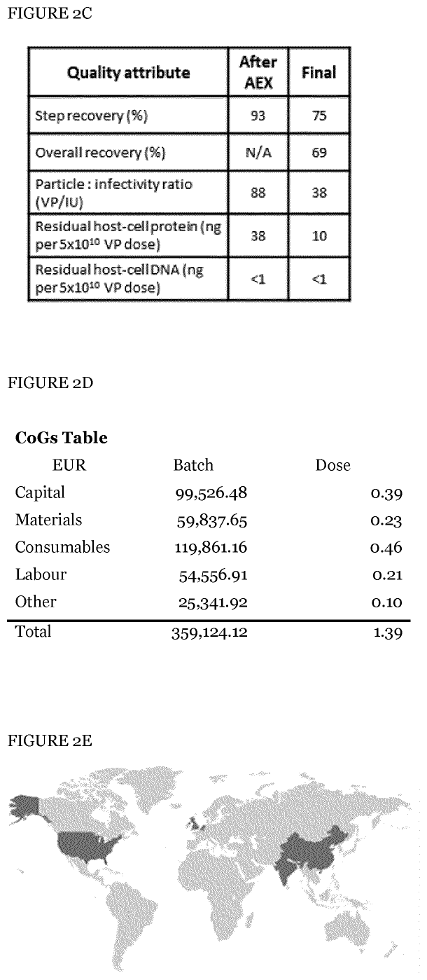

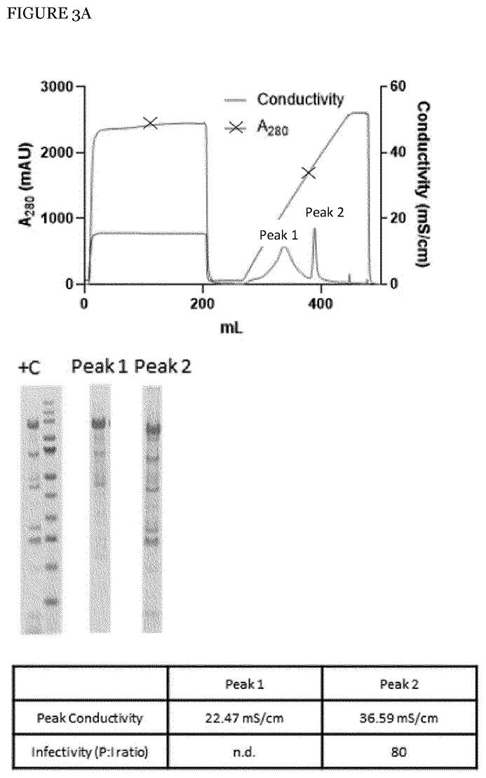

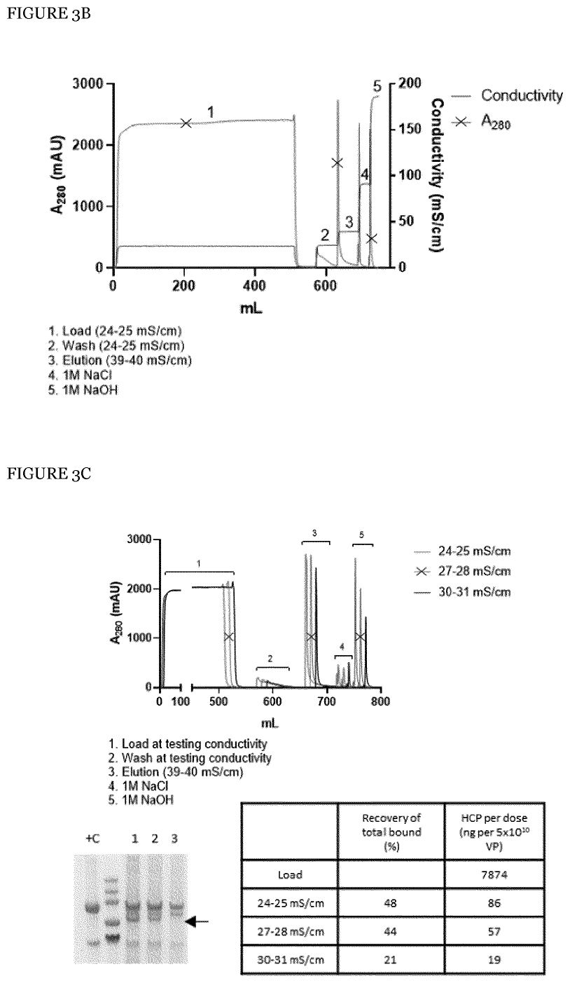

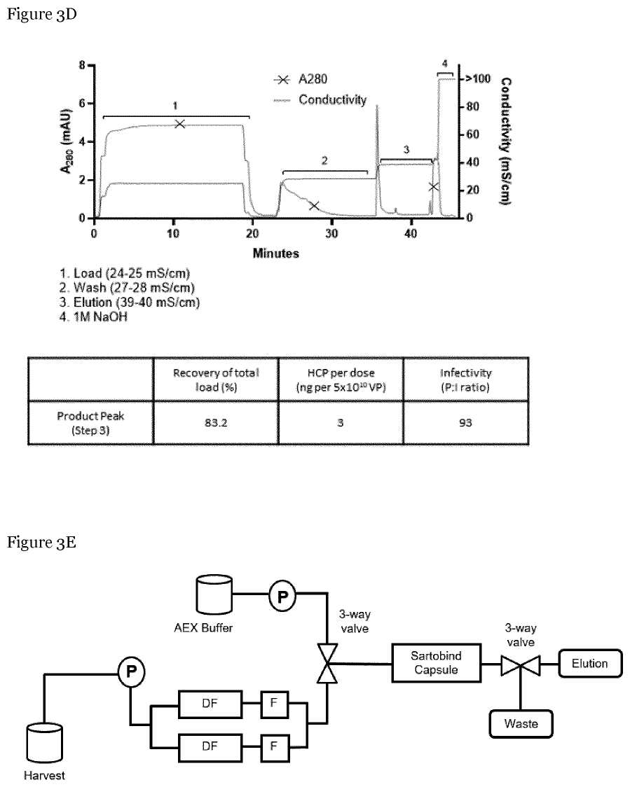

The inventors proceeded to develop the purification process in such a manner as to be scalable to 1000-2000 L. Handling of the large volumes of lysate, diafiltration buffer and waste during the initial TFF step was identified as a process bottleneck. We therefore developed a simplified DSP by loading the clarified lysate directly on an AEX membrane, followed by a single TFF diafiltration polish/formulation step. As well as removing the initial TFF step, this process change provides the option of execution of clarification and AEX as a single unit operation (FIG. 2A). Small-scale experiments were used to demonstrate feasibility and optimise conditions of the AEX step (FIG. 3 A-D), prior to execution of the complete revised DSP at 10 L scale (FIG. 2B). Binding capacity of the AEX membrane was reduced (to c. 3×10¹³ VP per mL of membrane, as compared to >5×10¹³ VP with loading of a diafiltered feed), but it was typically possible to recover c. 90% of loaded virus in the eluate. After a final TFF step, overall recovery was typically c. 70% and host cell protein and DNA were reduced to acceptable levels (FIG. 2C). The low levels of host cell DNA achieved, and the maintenance of acceptable binding capacity, were particularly surprising in view of the lack of a DNA precipitation step prior to the depth filter clarification. Efficient recovery of adenovirus from such a ‘direct load’ AEX has not, to the inventors' knowledge, previously been disclosed. The ability to achieve this enabled the simplification of the DSP from four steps to two or three. This efficient recovery with a ‘direct load’ AEX method is even more surprising given than no DNA precipitation step was performed before the depth filtration step. Without such a DNA precipitation step, it would be expected that the host cell DNA could interfere with the virus binding in the AEX, such that the host cell DNA bound competitively to the AEX membrane, and/or formed undesirable aggregates with the virus.

The simplicity and high productivity of the method of the invention are beneficial from an economic perspective. Process economic modelling of drug substance manufacturing suggests a cost of goods of <USD 2 per dose (FIG. 2D). Final prices may be somewhat higher due to additional costs, for example fill and finish.

### Example 3: Distributed Manufacturing

COVID-19 poses a unique challenge for vaccine manufacturing, both due to the volume and speed required, and due to concerns about equitable vaccine distribution and so-called ‘vaccine nationalism’. ‘Distributed manufacturing’ (manufacturing the same vaccine across multiple facilities in different countries) is a potential solution to these issues, but requires a readily transferable process (Gomez, P. L. and J. M. Robinson, Vaccine Manufacturing. Plotkin's Vaccines, 2018: p. 51-60.e1). Our process is well-suited to this strategy as it uses unit operations which are standard across the bioprocess industry, single-use product-contact materials throughout, and a viral vector of good biosafety (BSL1-2, dependent upon jurisdiction). Many contract manufacturing organisations (CMOs) are already equipped to execute such processes with little or no capital expenditure. We therefore pursued a distributed manufacturing strategy from an early stage of the pandemic. ChAdOx1 nCoV-19 (sometimes referred to as “AZD1222” drug substance) is now being manufactured at ≥1000 L scale in facilities in multiple countries (FIG. 2E).

We refer to FIG. 2 which shows Improved process, enabling cost-effective large-scale distributed manufacture

Panel A shows schematics of previous and revised DSPs. The dashed box indicates the feasibility of execution of depth filter clarification and AEX as a single unit operation. ‘Previous DSP’ means known DSP (i.e. not part of the invention). ‘Revised DSP’ means part of the invention.

Panel B shows chromatogram obtained when running ‘in-line’ depth filter clarification and AEX at 10 L scale. Absorbance at 280 nm is shown (line between 4-5 arbitrary units from 50-100 minutes), alongside conductivity (other line). For schematic of process skid, see FIG. 3E. A 150 mL Sartobind Q capsule was loaded at 3.8×10¹³ VP per mL of membrane. Numerals indicate stages: 1=loading, 2=wash, 3=elution, 4=1M sodium hydroxide sanitisation.

Panel C shows product recovery and quality from the 10 L process shown in Panel B and after final formulation by TFF.

Panel D tabulates modelled costs of drug substance production at 200 L scale. This includes capital for purchase of all equipment needed for the process; many facilities do not require this.

Panel E shows, in red, countries in which ChAdOx1 nCoV-19/AZD1222 is currently being manufactured.

We refer to FIG. 3 which shows small-scale optimisation of anion exchange with direct loading of clarified lysate.

Panel A shows gradient elution of ChAdOx1-luciferase from Sartobind Q anion exchange membrane. Filtered lysate containing 9×10¹³ VP of ChAdOx1-luciferase was loaded onto a 3 mL Sartobind nano Q capsule, followed by elution with a gradient of increasing salt concentration. Two peaks were observed (chromatogram) and analysed. Coomassie-stained SDS-PAGE, with comparison to virus purified by caesium chloride gradient ultracentrifugation (+C), shows that peak 1 contains impurities (notably free hexon protein) while Peak 2 contains virus. This was corroborated by infectivity assay. “n.d.” indicates “not detected”.

Panel B shows initial estimation of binding capacity and product recovery by step elution. Clarified lysate containing 2×10¹⁴ VP of ChAdOx1 nCoV-19 was loaded onto a 3 mL Sartobind nano Q capsule. Binding capacity and quantity of product bound was determined by collection of serial fractions of flowthrough during loading (step 1). After washing with equilibration buffer (step 2), product was eluted with a step to 39-40 mS/cm (step 3); steps 4 and 5 indicate regeneration with 1M NaCl and 1M NaOH respectively. Flowthrough and elution fractions were analysed by qPCR to calculate binding capacity and recovery: 10% breakthrough occurred at a load of 3.4×10¹³ VP per mL of bed volume; 90% of bound product was recovered.

Panel C shows optimisation of salt concentration/conductivity during loading and washing. Three runs were performed, in each of which filtered lysate containing 2×10¹⁴ VP of ChAdOx1 nCoV-19 was loaded onto a 3 mL Sartobind nano Q capsule, after adjustment of conductivity to the indicated values by addition of salt. Each run used wash buffer with conductivity matching the load. The chromatogram overlays results from the three runs. Eluates were analysed by Coomassie-stained SDS-PAGE, qPCR and HCP ELISA. Recovery and HCP are tabulated: the ‘basal’ condition (24-25 mS/cm) achieved nearly 2−log₁₀ reduction in HCP; increasing the conductivity of the load and wash achieved modest improvement in HCP clearance, with substantially reduced recovery with loading at 30-31 mS/cm. As virus in the flowthrough was not quantified, recovery is shown as % of loaded product (rather than bound product, as in panel B); the low recovery for the basal condition here thus reflects the overloading of the column.

Panel D shows initial results obtained using a 150 mL Sartobind Q capsule with relatively low load challenge. Clarified lysate containing 7.4×10¹⁴ VP of ChAdOx1 nCoV-19 was loaded, using conditions as indicated, based upon the results of the experiment shown in Panel C. The eluate was analysed by qPCR, HCP ELISA and infectivity assay. Recovery as % of loaded product, HCP and P:I ratio are tabulated.

Panel E illustrates system used for in-line clarification and anion exchange (P: pump; DF: depth filter; F: 0.2 μm filter), results of which are shown in FIG. 2B-C.

Viruses

The ChAdOx1 nCoV-19, ChAdOx1 Lassa-GP, ChAdOx1 luciferase and ChAdOx2 GFP vectors used here have previously been described¹⁻⁴. Virus used as seed to infect shake flask cultures and as standards in quality control assays was produced by caesium chloride density-gradient ultracentrifugation by the Jenner Institute Viral Vector Core Facility. Virus used as seed to infect 50 L and 200 L cultures was prepared using our previously described process in 3 L shake flasks or bioreactors, up to the point of the first tangential flow filtration (TFF) step 5. After this the concentrated and diafiltered lysate was aliquoted and frozen at −80° C.

Cells and Upstream Process

HEK293 T-rex cells (ThermoFisher) were banked and adapted to low-serum suspension culture in CD293 medium (ThermoFisher) as previously described 5. Cells were then adapted to increasing proportions of BalanCD293 medium (Fujifilm-Irvine Scientific), supplemented with 4 mM GlutaMAX (ThermoFisher), over one week.

For upstream process experiments, seed culture at 2× the specified final density was diluted by addition of 1 volume of fresh medium to reach the cell density specified for each experiment at the point of infection. A multiplicity of infection of 10 was used unless otherwise stated.

Each feed was with 0.05 volumes of BalanCD293 feed (Fujifilm-Irvine Scientific). Pre-infection, cultures were fed on the day cell density exceeded 1×10⁶ cells/mL. Cultures for which the intended cell density at the point of infection was ≥3×10⁶ cells/mL received a second pre-infection feed when cell density exceeded 4×10⁶ cells/mL. Post-infection, all cultures were fed at 0.5 h and 22 h after infection.

Shake flask experiments were performed in Erlenmeyer flasks (Corning), with a working volume of 25-35 mL in a 125 mL flask unless otherwise stated. BioBlu 3c and 14c (Eppendorf) single-use bioreactor vessels were used in accordance with the manufacturers' instructions. A GX bioreactor controller unit and C-BIO software (both from Global Process Control) were used to control both vessel types. Dissolved oxygen (DO) was regulated at a setpoint of 55% air saturation by addition of medical air via macrosparger. pH was regulated in the range 7.2-7.3 as previously described 5.

50 L and 200 L upstream processes were performed using Pall Allegro stirred tank reactors (STRs).

Unless otherwise stated, bioreactors were seeded at 0.4-0.6×10⁶ cells/mL in c. 35% of the maximum working volume. Antifoam C emulsion (SigmaAldrich) was used in 50 L and 200 L STRs. 0.05 culture volumes of BalanCD feed was added when the density reached 1.0×10⁶ culture cells/mL. At a cell density of 4.0×10⁶/mL (range 3.0-6.0×10⁶), cells were diluted with 1 volume of medium and infected, using an MOI of 10 unless otherwise stated. 0.05 volumes of BalanCD feed were added 30 minutes after infection, and again after 22 hours.

Lysis, Nucleic Acid Digestion and Clarification

Lysis was performed as previously described⁵, in the culture vessel, with the exception that the concentration of Benzonase (MerckMillipore) was reduced to 15 units/mL. No DNA precipitation step was performed before the clarification by depth filtration. Lysis was initiated at 42-48 h after infection, with the exception of the productivity kinetic experiments shown in FIGS. 1C-D. Two hours after addition of lysis buffer, clarification was initiated, using Millistak+® HC Pro CoSP depth filters as in our previous work 5. During 200 L runs, an Advanced MVP skid (Pall Biotech) was used for filtration steps.

Tangential Flow and Bioburden Reduction Filtration

Tangential flow filtration was performed essentially as we have previously described 5, scaled appropriately and with the following modifications. Where TFF was performed before anion exchange (AEX), i.e. for the 200 L run producing product as reported in FIG. 1G, only 2-fold concentration was performed, prior to 6 diavolumes of diafiltration. For TFF after AEX, Omega T-series 300 kDa cut-off flat sheet filters (Pall Biotech) were used. For TFF during 200 L runs, an Allegro CS 4500 single-use TFF skid was used (Sartorius). A Supor EKV 0.2 μm filter was used for bioburden reduction filtration after the final TFF.

Anion Exchange Chromatography

Where preceded by TFF (run reported in FIG. 1G), AEX was performed as previously reported 5, with scaling of the chromatography capsule and buffer volumes based upon anticipated binding capacity of 7×10¹³ VP per mL of membrane volume.

For ‘direct-load’ AEX (loading clarified lysate), small-scale studies (FIG. 3) were performed using an Akta Pure (GE) and 3 mL bed volume/8 mm bed height SartobindQ Nano capsules (Sartorius). Equilibration buffer comprised 20 mM Tris-HCL pH8.0, 1 mM MgCl₂, 0.1% v/v polysorbate 20, 5% w/v sucrose; this was also used as a base for wash buffers. Elution buffer comprised 20 mM Tris-HCL pH8.0, 1 mM MgCl₂, 0.1% v/v polysorbate 20, 5% w/v sucrose, 600 mM NaCl, except where salt concentration was varied, as stated. Adjustment of the conductivity of the sample and wash buffers, to target values as stated in the descriptions of individual experiments, was performed using 5M NaCl (Sigma).

Column equilibration was in accordance with the manufacturer's instructions. After loading, capsule was washed with 10 MV of equilibration buffer before elution step (both at 5 MV/min).

For the ‘direct-load’ AEX purification from a 10 L bioreactor (FIG. 2B) a peristaltic pump-driven rig was constructed, as shown in FIG. 3E, incorporating a CoSP depth filter (as above), Millipak-20 0.2 μm filter, and 150 mL/8 mm bed height Sartobind Q capsule (Sartorius), plus single-use UV absorbance, conductivity and pressure sensors (Pendotech). Buffers, column equilibration, sample loading, washing and elution were as described above, with the exception that a flow rate of 0.7 membrane volumes/minute was used for sample loading, washing and elution.

Product Quantification and Assessment of Product Quality

Product quantification was as previously reported, using qPCR and UV spectrophotometry assays for viral particles in impure and pure samples respectively, and an immunostaining-based infectivity assay 5.

Residual host-cell protein (HCP) was quantified using the HEK293 HCP ELISA kit (Cygnus Technologies), according to the manufacturer's instructions. Residual host cell DNA was quantified using a previously reported quantitative PCR method targeting a 94 base pair amplicon within the Alu repeats⁶. The lower limit of quantification was 100 pg/mL for intact HEK293 DNA.

### REFERENCES TO EXAMPLES 1, 2 AND 3

1 van Doremalen, N. et al. ChAdOx1 nCoV-19 vaccine prevents SARS-CoV-2 pneumonia in rhesus macaques. Nature, doi:10.1038/s41586-020-2608-y (2020).
2 Purushotham, J., Lambe, T. & Gilbert, S. C. Vaccine platforms for the prevention of Lassa fever. Immunol Lett 215, 1-11, doi:10.1016/j.imlet.2019.03.008 (2019).
3 Dicks, M. D. et al. Differential immunogenicity between HAdV-5 and chimpanzee adenovirus vector ChAdOx1 is independent of fiber and penton RGD loop sequences in mice. Sci Rep 5, 16756, doi:10.1038/srep16756 (2015).
4 Morris, S. J. S., Sarah; Spencer, Alexandra J.; Gilbert, Sarah C. Simian adenoviruses as vaccine vectors. Future Virology 11, 649-659, doi:10.2217/fvl-2016-0070 (2016).
5 Fedosyuk, S. et al. Simian adenovirus vector production for early-phase clinical trials: A simple method applicable to multiple serotypes and using entirely disposable product-contact components. Vaccine, doi:10.1016/j.vaccine.2019.04.056 (2019).
6 Zhang, W. et al. Development and qualification of a high sensitivity, high throughput Q-PCR assay for quantitation of residual host cell DNA in purification process intermediate and drug substance samples. J Pharm Biomed Anal 100, 145-149, doi:10.1016/j.jpba.2014.07.037 (2014).

### Example 4: 50 L Stirred Tank Bioreactor Production & Purification of ChAdOx1 nCoV-19

A 40 L culture was infected and harvested in a 50 L STR, as previously described (Fedosyuk et al 2019 Vaccine vol 37 pages 6951-6961 (doi: 10.1016)), with the exception of the use of the BalanCD293 medium/feed strategy, with a starting cell density of 2e6 cells/mL (diluted from 4e6 cells/mL) and an MOI of 10.

The product was subsequently purified using a strategy as previously described (Fedosyuk et al 2019 Vaccine vol 37 pages 6951-6961 (doi: 10.1016)). The final purified product yield was 1.8×10¹¹ VP per mL of the upstream process (FIG. 1 E-G).

### Example 5: Attainment of High Purity and Recovery by Simplified Downstream Process

We refer to FIG. 2 and FIG. 3.

### Example 6: Comparative Data

An important advantage of the method of the invention (for example with direct AEX loading of clarified lysate) is that it allows reduction of the complexity of the purification process (‘downstream process’) to two or three unit operations (as shown in FIGS. 2A & 3E). This is compared to known methods which require four unit operations (as shown in FIG. 2A). We refer also to the disclosure of Fedosyuk et al 2019 (Vaccine vol 37 pages 6951-6961) FIG. 5A—it should be noted that this known Fedosyuk process also contains a fourth [clarification] step before the three steps shown in FIG. 5A of Fedosyuk et al.

A further advantage of the method of the invention is higher product recovery compared to known methods. This is supported by the AEX step recovery data shown in FIGS. 2C and 3A/D (80, 83, 93%). (It should also be noted that the lower figures in FIG. 3C are not representative of the achievable recovery as the columns were over-loaded in this example.) This may be compared to Fedosyuk et al 2019 (Vaccine vol 37 pages 6951-6961) Table 2 which shows that up to 20% loss may occur in the TFF1 step of the known method. By comparison, this known TFF1 step has been eliminated from the method of the invention.

A further advantage which may be ascribed to the method of the invention is that losses on the known AEX step were greater than they are on the AEX step of the method of the invention. For comparison, we refer to Fedosyuk et al 2019 (Vaccine vol 37 pages 6951-6961) Table 2 compared to the recovery on the new AEX shown in the Figures herein.

Although illustrative embodiments of the invention have been disclosed in detail herein, with reference to the accompanying drawings, it is understood that the invention is not limited to these precise embodiments and that various changes and modifications can be effected therein by one skilled in the art without departing from the scope of the invention as defined by the appended claims and their equivalents.

1. A method for purifying an adenovirus comprising
(a) providing a liquid sample comprising adenovirus
(b) clarifying said sample by depth filtration
(c) performing anion exchange chromatography comprising the steps of
(i) directly applying the clarified sample of (b) to anion exchange
(ii) eluting adenovirus from the anion exchange to provide an eluate.

2. A method according to claim 1 wherein the anion exchange chromatography of (c) comprises membrane anion exchange chromatography or column anion exchange chromatography.

3. A method according to claim 2 wherein the membrane comprises quaternary amines.

4. A method according to claim 2 or claim 3 wherein the membrane comprises Sartobind® Q membrane.

5. A method according to any preceding claim wherein the anion exchange chromatography is carried out with load and wash salt concentrations in the range 24 to 31 mS/cm, with 20 mM Tris pH 8.0.

6. A method according to any preceding claim wherein the anion exchange chromatography is carried out with load and wash salt concentrations in the range 280 mM NaCl to 361 mM NaCl, with 20 mM Tris pH 8.0.

7. A method according to any preceding claim wherein the anion exchange chromatography is carried out with wash conditions of 15 mS/cm to 30 mS/cm conductivity, with 20 mM Tris pH 8.0.

8. A method according to any preceding claim wherein the sample is adjusted to a conductivity of 28 mS/cm before applying to the anion exchange column.

9. A method according to any preceding claim wherein said depth filtration of step (b) comprises primary clarification followed by secondary clarification.

10. A method according to any preceding claim wherein said depth filtration of step (b) comprises combined primary and secondary clarification.

11. A method according to any preceding claim wherein said depth filtration of step (b) comprises use of a CoSP depth filter.

12. A method according to any preceding claim wherein the anion exchange chromatography of (c) is performed in-line with the clarifying depth filtration of (b) as a single unit operation.

13. A method according to any preceding claim wherein said liquid sample comprises cell lysate produced from cultured host cells comprising adenovirus.

14. A method according to claim 13 wherein said cell lysate is produced by treating a sample of cultured host cells comprising adenovirus with a detergent.

15. A method according to claim 13 or claim 14 wherein said cell lysate is produced by treating a sample of cultured host cells comprising adenovirus with a nuclease.

16. A method according to claim 15 wherein said nuclease comprises DNAse and/or RNAse.

17. A method according to claim 15 or claim 16 wherein said nuclease comprises, or consists of, endonuclease from Serratia marcescens.

18. A method according to any of claims 15 to 17 wherein said nuclease comprises, or consists of, Benzonase®.

19. A method according to claim 18 wherein said Benzonase® is added to said sample at a final concentration of 15 units/millilitre.

20. A method according to any preceding claim further comprising:
(d) performing buffer exchange on the eluate of (c).

21. A method according to claim 20 wherein step (d) comprises tangential flow filtration.

22. A method according to any preceding claim wherein said adenovirus is, or is derived from, a simian adenovirus, preferably wherein said adenovirus is, or is derived from, a species E simian adenovirus, preferably wherein said adenovirus is ChAdOx1.

23. A method according to claim 22 wherein the adenovirus is ChAdOx1 nCoV-19.

24. A method according to any of claims 1 to 21 wherein said adenovirus is an adenovirus having a capsid with charge characteristics similar to those of ChAdOx1.

25. A method according to any of claims 1 to 21 wherein said adenovirus is an adenovirus having capsid charge characteristics such that its elution conductivity, in 20 mM Tris pH 8 on a Sartobind Q or Pall Mustang Q membrane chromatography unit, exceeds 25 mS/cm.

26. An adenovirus prepared by a method according to any preceding claim.

27. A composition comprising an adenovirus according to claim 26.

28. A composition according to claim 27 which is a pharmaceutical composition.

29. A composition according to claim 27 which is a vaccine composition.
